(19) United States
(12) Patent Application Publication
(10) Pub. No.: US 2012/0089206 A1
(43) Pub. Date: Apr. 12, 2012

(54) Device using light energy to excite brain waves and method using the same

(75) Inventor: Chi-Hua Wu, Taipei (TW)

(73) Assignee: UNITED INTEGRATED SERVICES CO., LTD.

(21) Appl. No.: 12/925,012

(22) Filed: Oct. 12, 2010

(51) Int. Cl. A61N 5/06; A61N 5/067

(52) U.S. Cl. 607/89; 607/88

(57) ABSTRACT

A method using light energy to excite brain waves and method using the same employ a light-power light source module in collaboration with a control module to apply a non-intrusive light energy stimulation at a specific frequency to users' skin so as to generate potential variation of brain wave of users. The device can be operated at different frequencies to stimulate nerves under skin and further affect the potential of brain waves, thereby improving functional imbalance such as anxiety, tension, uneasiness and insomnia.

### BACKGROUND OF THE INVENTION

1. Field of the Invention

The present invention relates to a device using light energy to excite brain waves and method using the same, and more particularly to a device and a method using low-power light with specific frequencies to irradiate skin, transmit excited neural signals through a neural network under skin to the brain, and excite brain waves having the specific frequencies.

2. Description of the Related Art

The concept of brain waves was brought up by a German psychologist, Dr. Freda Hansburg in 1929. He inferred that brains of human being have voltage variation after observing electricity generated by electric eels. Brain wave voltage is below 100 μV, normally several tens of μV, and in a frequency range of 0.1˜40 Hz. Variables associated with brain voltage variation have amplitude, frequency and phase. There are five brain waves categorized in terms of frequency, namely:

1. δ wave: It is a low-speed and high-amplitude brain wave in a frequency range of 0.5˜4 Hz and is relevant to stage 3 and stage 4 of the Slow Wave Sleep. Generally, the δ waves are treated as the brains wave in deep sleep. When the δ waves dominate, human body is in an unconsciously deep sleep state, and a dreamless and unconscious condition usually directly affects sleep quality in a positive way.

2. θ wave: It is a brain wave in a frequency range of 4˜8 Hz, governs subconscious mind, and affects involuntary attitude, expectation, belief, behavior and the like, such as hypnosis and meditation.

3. α wave: It is a low-speed wave in a frequency range of 8˜13 Hz and can be further classified as α1 and α2. When the α waves dominate, people are conscious and the bodies are in a relaxed condition. The α waves are reduced when people are exercising or anxious. As for therapy, α waves can be used to treat mental health problems, such as anxiety and tension.

4. β wave: It is a fast-speed and low-amplitude wave in a frequency range of 13˜30 Hz and is a brain wave appearing when people are conscious. The β waves are involved with thinking, anxiety, computing and attention. When the high-frequency β waves appear, people are emotionally excited or anxious.

5. γ wave: It is a high-frequency wave above 30 Hz. Given the classification of brain waves, different brain voltages (different frequencies) resulting from different types of brain waves affect human body in different ways. According to research, the best learning condition occurs when brain waves have a frequency range between that of the α waves and the β waves, and the best condition before sleep occurs when brain waves have a frequency range between the θ waves and the α waves.

Actually, the foregoing brain waves can be induced from external stimulation. Clinically, the external stimulation generating brain voltage includes three types, that is, sense of sight, sense of hearing and sense of body. Qigong, a deep breathing exercise, and acupuncture can also affect brain waves. Measurements of brain waves of people after Qigong exercise show that Qigong affects the α1, α2, θ and δ waves. A research studying how Qigong affects the α waves of an archer from stretching the bow to releasing the arrow shows that when the power values of the α waves in the left frontal lobe, the left parietal and the left temporal lobe of the brain of the archer increase, the score is better than that in a normal condition. A research indicates that brightness contrast affects physiological reactions and brain waves. When the brightness contrast is low, the θ waves in the frontal lobe are more active and the α waves are suppressed more, reflecting that attention is more focused and working memories are more active. A research points out that people have higher energy of the δ waves when listening to music than not listening to any music at all, and the influence of music upon the δ waves is significant, proving that sensory stimulation and selective attention affects the intensity of the δ waves of the testees and also discovering that musical preference factor affects the intensity of the α waves. A continuous wavelet transform (CWT) technique is employed to analyze the brain waves after needling the Waiguan point of a left hand and realize it is statistically meaningful after comparing the brain waves before and after the needling. A research discovered that practicing strength of fingers changes the activity of brain's cortex. Measurements also show that brain waves are significantly varied after the Neiguan point is acupunctured. The energy intensity of the α waves escalates and brain voltages during acupuncture and after acupuncture are even higher. One research finds that low-frequency musical stimulation has noticeably small value measured in P3 location as shown in FIG. 5. One research discovered that brain voltages increase after laser acupuncture.

With the foregoing research data, the α waves correlate with mind relaxing. If the α waves can be excited or intensified, it is helpful to improve functional imbalance of human body such as anxiety, tension, restlessness or insomnia.

Light therapy plays a critical role in modern medicine. Light sources for light therapy are classified into laser and light-emitting diode. Low Level Laser Therapy (LLLT) has attracted attention since 1970s, various applications thereof emerge, and considerable development has been achieved on the aspects of wound care, wound healing, pain relief and biological stimulation before and after operation. Using Laser Evoked Potential (LEP), which generates temporary electrical change when an external factor stimulates a nervous system, to test signal transmission paths is one of the important subjects. A literature in 1985 has indicated that a LEP can be measured from the Erb's point of the neck by illuminating low-energy helium-neon laser to a wrist. Lately, laser oftentimes serves as a stimulus to study transmission paths of a neural system in response to pain, and the testing points have been moved to a brain. However, according to the aforementioned literature, tools for measuring LEP are mainly the laser stimuli that inevitably cause pain.

Despite many techniques or methods capable of affecting brain waves, uneasiness and fear may arise from their operations, for example, the intrusive acupuncture or the pain-causing laser stimuli. Under the premise of causing no uneasiness and pain, how to address a feasible solution to effectively excite user's brain waves needs to be further explored.

### SUMMARY OF THE INVENTION

A first objective of the present invention is to provide a method using light energy to excite brain waves, which employs low-power light energy at a specific frequency to stimulate nerve receptors under skin and excite the brain and generate brain waves having a specific frequency through a neural network so as to further improve functional imbalance of users associated with anxiety, tension, uneasiness and insomnia.

To achieve the foregoing objective, the method using light energy to excite brain waves, comprising steps of:

providing a light source module having at least one photoelectric element and generating a light energy having a frequency controlled within a frequency range of brain waves of a user;

illuminating the light energy of the at least one photoelectric element of the light source module on the user's skin for a period of time; and

generating a stimulating signal of the light energy on the user's skin, receiving the stimulating signal by nerve receptors of the skin, transmitting an excited neural signal corresponding to the received stimulating signal to a brain of the user through a neural network, and generating a resonance in the brain to excite the brain waves of the user in the frequency range.

As employing light energy to stimulate skin in generation of a stimulation signal to user's skin and transmit an excited neural signal corresponding to the stimulation signal to the brain to generate resonance after the signal is received by nerve receptors under skin, the method of the present invention is completely non-intrusive to human body. Besides, the light irradiation is not painful due to low power, thereby causing no fear and uneasiness of users during operation.

A second objective of the present invention is to provide a device using light energy to excite brain waves, which illuminates low-power light energy on users' skin to transmit excited neural signals to the brain through a neural network so as to excite brain waves.

To achieve the foregoing objective, the device using light energy to excite brain waves has a light source module and a control module.

The light source module has a bearing member and at least one photoelectric element. The at least one photoelectric element is mounted on the bearing member and has a lighting end. The control module has a controller and at least one driving circuit. The controller has an input terminal and an output terminal. Each one of the at least one driving circuit has an input terminal and an output terminal. The input terminal is connected with the output terminal of the controller. The output terminal is connected with the at least one photoelectric element and drives one of the at least one photoelectric element to generate light energy.

The light source module is mounted on users' skin through the bearing member. The lighting end of each photoelectric element faces users' skin and illuminate low-power light to generate light energy stimulating signals, transmit excited neural signals corresponding to the stimulating signals to the brain through the neural network under the skin and generate resonance and excite brain waves.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a device using light energy to excite brain waves in accordance with the present invention;

FIG. 2 is an operational perspective view of the device using light energy to excite brain waves in FIG. 1;

FIG. 3A is a circuit diagram of a first embodiment of a control module of the device using light energy to excite brain waves in FIG. 1;

FIG. 3B is a circuit diagram of the control module of the device using light energy to excite brain waves in FIG. 1;

FIG. 4 is a block diagram of a second embodiment of a control module of the device using light energy to excite brain waves in FIG. 1;

FIG. 5 is a schematic diagram illustrating locations of electrodes attached on a scalp when testing in accordance with the present invention;

FIG. 6 is a curve diagram of energy of α waves measured from a location P3O1 of the scalp of 40 subjects when tested in accordance with the present invention; and

FIG. 7 is a curve diagram of energy of α waves measured from another location P4O2 of the scalp of 40 subjects when tested in accordance with the present invention.

### DETAILED DESCRIPTION OF THE INVENTION

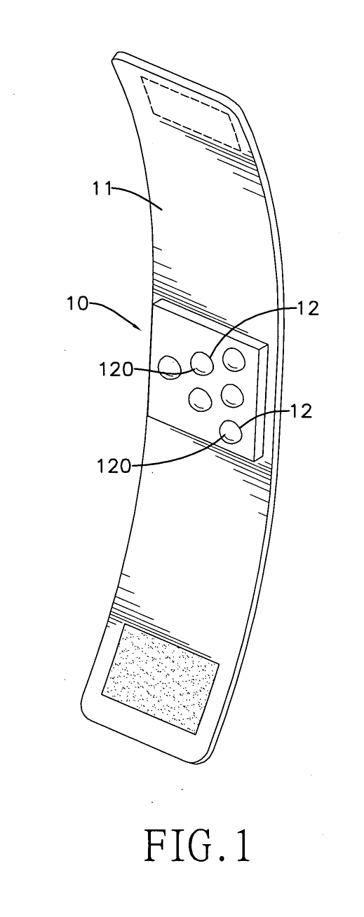

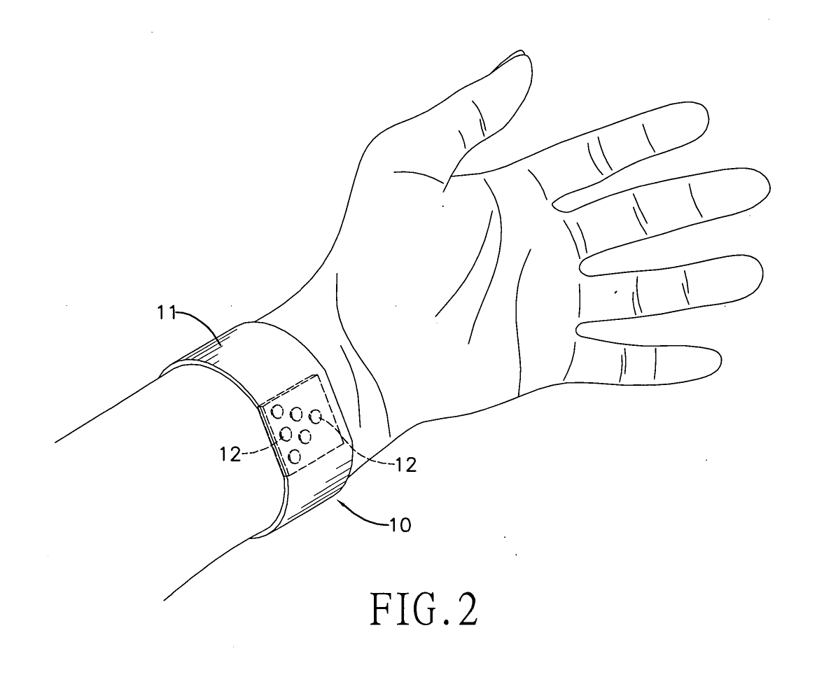

With reference to FIG. 1, a device using light energy to excite brain waves in accordance with the present invention has a light source module 10 and a control module. The light source module 10 has a bearing member 11, at least one photoelectric element 12. The bearing member 11 is slender and takes a form of a wristband, may serve as a palm band or an arm band for a female user whose wrist size is small, and has two ends connected by a connection assembly such as snap fasteners, fabric hook-and-loop fasteners, such as Velcro band fasteners or the like. The bearing member 11 has an inside surface and an outside surface. The at least one photoelectric element 12 is centrally mounted on the inside surface and may be a laser diode, a near-infrared diode laser or a light-emitting diode (LED). In the present embodiment, the laser diode is employed, and six photoelectric elements 12 are centrally mounted on the inside surface of the bearing member 11, are longitudinally arranged as three rows respectively having one, two and three photoelectric elements 12 in a form of an equilateral triangle. The bottom side of the equilateral triangle is formed by the three photoelectric elements and each two adjacent photoelectric elements are equally spaced by a distance, for example, 2 cm in the present embodiment. Hence, the length of the bottom side is 4 cm. Given the two equal sides being 3 cm respectively, the height of the equilateral triangle is \sqrt{3²−2²}=2.236 cm. Each photoelectric element 12 has a lighting end 120 facing skin of a user. Preferably, the skin is located on a palm, or on an inner side of an arm. As the bearing member 11 of the present embodiment takes a form of a wrist band, the bearing member 11 is mounted around a wrist. With reference to FIG. 2, when the light source module 10 is sleeved around a wrist by using the connection assembly. The lighting end of each photoelectric element 12 mounted on the inside surface of the bearing member 11 faces smooth skin of an inner side of an arm. Controlled by the control module, the photoelectric element 12 generates a low-power light and is operated in a specific frequency range. In the present embodiment, a power of the photoelectric element 12 is 7 mW˜30 mW. A dose is 10˜30 Joule/cm². An optical wavelength is 700˜940 nm. An operating frequency range is 1˜20 Hz. A driving signal of the control module is in a form of a square wave and a duty cycle thereof is 50%.

As mentioned earlier, the photoelectric element 12 employs laser diode to generate invisible laser light with low energy. The so-called low-energy laser specifies a laser with an output power less than 500 mW and a dose less than 35 Joule. Such energy is not sufficient yet to destroy hydrogen bonds or any Van Der Waals bonds in tissue, and thus causes no change of organization and results in photochemical effect.

Type of laser (wavelength), dose and illumination time and intensity vary depending on purpose and the person who is being treated. It is generally considered that high-intensity stimulation is harmful to organization while low-intensity stimulation is effective in generating excitement. Low-energy laser mainly serves to excite biological cells and induces or strengthens certain biological reactions with adequate applied energy. In addition to the low-energy laser, near-infrared light or light-emitting diode can also serve to illuminate and stimulate users' skin in the form of square waves with an operating frequency range of 4˜12 Hz so as to further generate brain voltage variation under the premise of causing no pain to users.

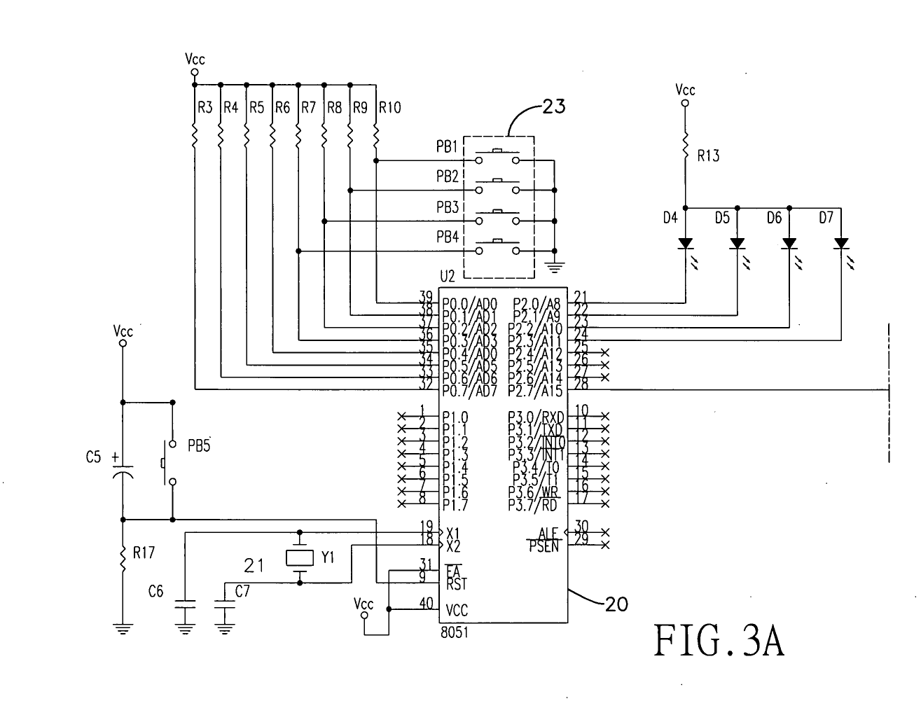

The control module can be also mounted on the bearing member 11. With reference to FIGS. 3A and 3B, a feasible circuit of the control module has a controller 20 and at least one driving circuit 22. The number of the driving circuit 22 corresponds to that of the photoelectric element 12 in the light source module 10 for respectively driving the at least one photoelectric element 12. Therefore, the light source module 10 has six photoelectric elements, so six driving circuit are required (only two of the driving circuit are shown in FIG. 3B).

In the present embodiment, the controller 20 is a microcontroller unit (MCU) whose output terminal is connected with the driving circuits 22. An output terminal of each driving circuit 12 is connected with the one of the at least one photoelectric element 12 of the light source module 10. The controller 20 is externally connected with an oscillator 21 to generate different operating frequencies by oscillation. The controller 20 is embedded with a timer and a task mode switching process to provide various task modes for user to select. The feasible task modes include but not limited to the following:

1. Each one of the at least one photoelectric element 12 is instructed to continuously illuminate low-power light for a period of time at a specific frequency. For example, the photoelectric element 12 is instructed to continuously illuminate for 10 minutes to generate low-power light at 10 Hz.

2. The photoelectric element 12 is instructed to illuminate low-power light at a specific frequency for a period of time and then convert to another frequency and continuously illuminate a period of time. For example, each one of the at least one photoelectric element 12 is instructed to continuously illuminate for 10˜20 minutes, switch frequency to 12 Hz and continuously illuminate for 5˜10 minutes. Alternatively, the photoelectric element 12 is instructed to generate low-power light at 10 Hz, continuously illuminate for 5˜10 minutes, then switch frequency to 12 Hz and continuously illuminate for 5˜10 minutes.

In collaboration with the foregoing selection of task modes, an input terminal of the controller 20 is further connected with an input unit 23 which is composed of multiple press buttons or switches for users to power on/off, set up/select a specific task mode.

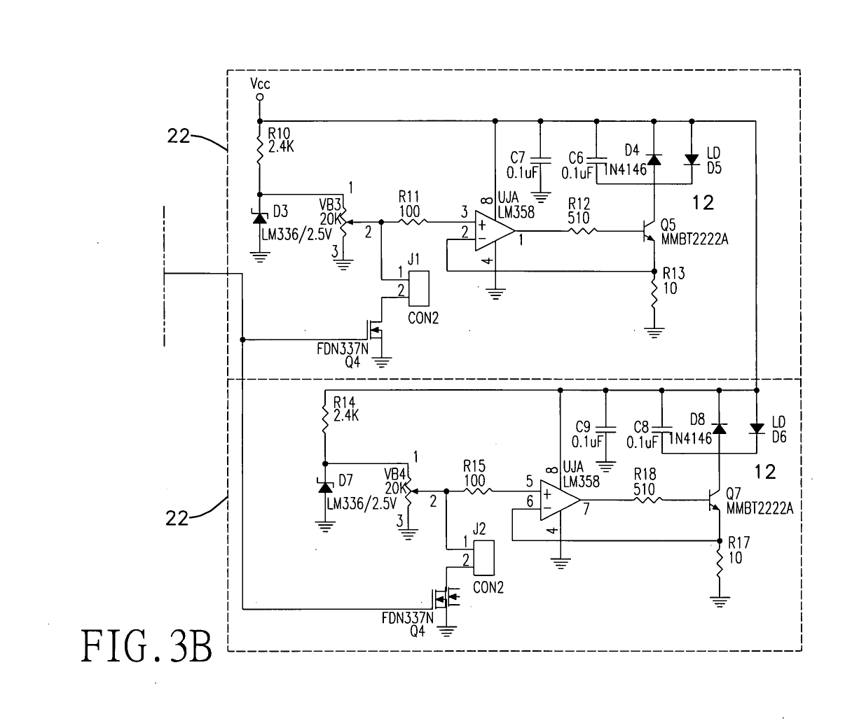

With reference to FIG. 4, an output terminal of the controller 20 is connected with a display driving circuit 24, and is connected with a display through the display driving circuit 24. A display 25 can be used to display various data and function options and constitutes a touch panel by incorporating the input unit 23.

The display 25 can also serve to generate 7-color back light and display color light with different wavelength, such as blue (470 nm), and yellow or orange (560 nm) as the background color.

Detailed description of a method using light energy to excite brain waves in accordance with the present invention is as follows:

The device using light energy to excite brain waves is fixed on a user's arm adjacent to a wrist, as shown in FIG. 2, by the bearing member 11, so that the lighting end 120 of each one of the at least one photoelectric element 12 mounted on the inside surface of the bearing member 11 faces to soft skin on the inner side of the arm. Besides the soft skin on the inner side of an arm, a palm is also an ideal place to apply the stimulation; in other words, each one of the at least one photoelectric element illuminates the palm.

When each one of the at least one photoelectric element 12 of the light source module 10 generates low-power light at a specific frequency and irradiates the light onto the skin to generate light energy stimulating signals, the light energy stimulating signals are sensed by nerve receptors under the skin. Specifically, the nerve receptors sensing the stimulation signals are the Meissner Corpuscles on skin. The Meissner Corpuscles are vibration receptors at lower frequency (approximately 20˜40 Hz) and are located on the dermis layer of the smooth skin.

After the Meissner Corpuscles sense the stimulating signals, excited neural signals corresponding to the stimulating signals are transmitted to the brain through a neural network. The brain generates a resonance to excite specific brain waves. For example, the brain is excited to generate the α waves when an operating frequency of the photoelectric element 12 is at 10 Hz, the θ waves when an operating frequency of the photoelectric element 12 is 6 Hz, and the high-frequency α waves when an operating frequency of the photoelectric element 12 is 12 Hz.

As mentioned, the control module has a task mode selection function. Hence, users can vary brain potentials by switching task modes and further alter mental conditions accordingly. Listed below are the examples:

First excite the α1 waves (10 Hz) of the brain and then modulate frequency to excite the α2 waves (12 Hz) so that users can first relax and then turn to focus. This mode facilitates users to focus attention to enhance learning efficiency.

Further or first excite the α1 waves (10 Hz) and then modulate frequency to excite the θ (6 Hz) waves so that users first enter a relaxing state in turn inducing drowsiness. This mode helps users to sleep soundly and to solve the insomnia problem.

Actual tests using light energy to excite brain waves and test results are presented as follows:

(a) Subject: There are 40 Volunteers (age range: 18˜30), classified as a test group, in total 20 persons, and a control group, in total 20 persons, which are randomly assigned.

(b) Exclusion condition:

1. Having specific diseases such as cardiovascular disease and the like and history thereof.

2. Currently taking any medicine, including analgesic, antiphlogistic or diuretic.

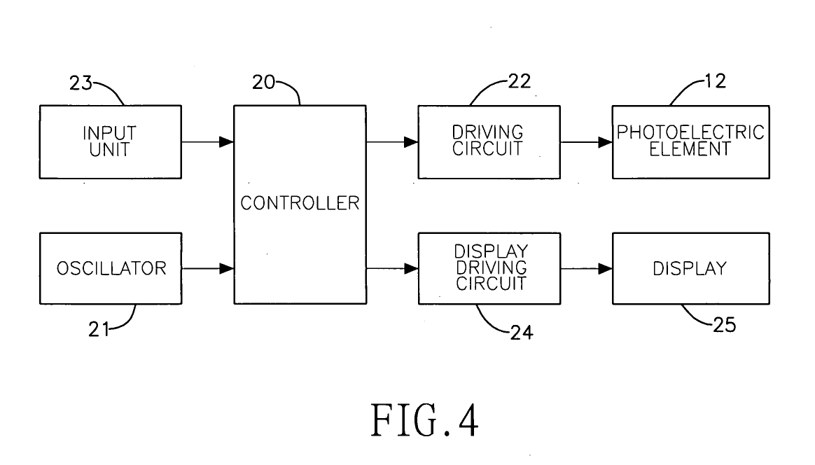

(c) Test method: Record variation of electroencephalography (EEG) by attaching 20 electrodes distributed as shown in FIG. 5. The recording instrument is Neurofax EEG-1000, 6.0 version, made by Nihon Kohden Corporation in Japan.

(4) Test procedures:

1. After the subject opens eyes and sits still for 5 minutes, start measuring EEG.

2. After measuring EEG for 5 minutes, use a laser having a frequency 10 Hz to illuminate a palm of the subject for 10 minutes. Then, measure EEG for 15 minutes.

3. Use the laser to illuminate each member of the test group, while using a false laser output (the laser having no output) to illuminate each member of the control group.

(5) Test result:

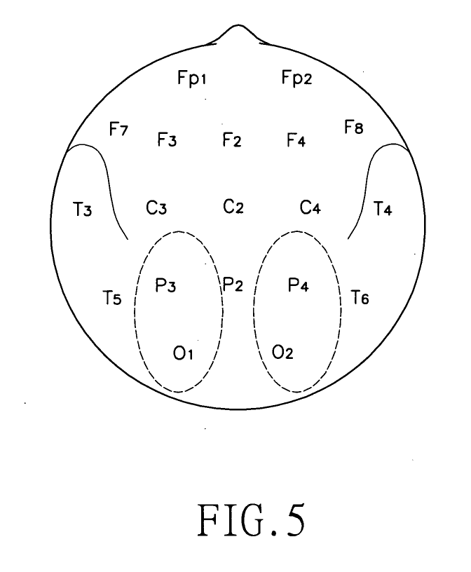

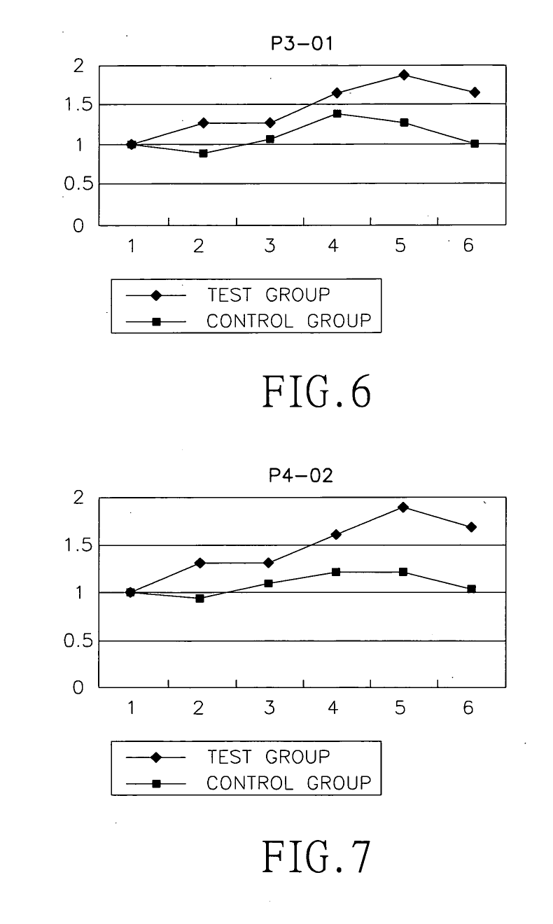

As the excitation light source is operated at the frequency 10 Hz, brain waves of the subjects are analyzed in a frequency range of 9.5˜10.5 Hz. With reference to FIG. 6, energy variation of α waves associated with P₃ and O₁ on scalps of subjects and measured every 5 minutes is shown. With reference to FIG. 7, energy variation of the α waves associated with P₄ and O₂ on scalps of subjects and measured every 5 minutes is shown. The curves marked by diamonds pertain to the energy variation of the α waves of the test group subjected to the stimulation source. The curves marked by squares pertain to the energy variation of the α waves of the control group subjected to no stimulation source. From the tests, the energy of the α waves on scalp locations P₃, O₁, P₄ and O₂ is noticeably stimulated and shows an increasing trend. In contrast, the energy of the α waves of the test group subjected to the stimulation source is much higher than that of the control group subjected to no stimulation source.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

What is claimed is:

1. A method using light energy to excite brain waves, comprising steps of:
providing a light source module having at least one photoelectric element and generating a light having a frequency controlled within a frequency range of brain waves of a user;
illuminating the light of the at least one photoelectric element of the light source module on the user's skin for a period of time; and
generating a stimulating signal of the light on the user's skin, receiving the stimulating signal by nerve receptors of the skin, transmitting an excited neural signal corresponding to the received stimulating signal to a brain of the user through a neural network, and generating a resonance in the brain to excite the brain waves of the user in the frequency range.

2. The method as claimed in claim 1, wherein the frequency is in a frequency range of α waves.

3. The method as claimed in claim 1, wherein the light energy illuminated by the light source module is operated at a first frequency for a period of time, and then is continuously operated at a second frequency for a period of time.

4. The method as claimed in claim 3, wherein the first frequency is in a frequency range of α₁ waves, and the second frequency is in a frequency range of α₂ waves.

5. The method as claimed in claim 3, wherein the first frequency is in a frequency range of α waves, and the second frequency is in a frequency range of θ waves.

6. A device using light energy to excite brain waves, comprising:
a light source module having:
a bearing member;
at least one photoelectric element mounted on the bearing member and having a lighting end; and
a control module having:
a controller having an input terminal and an output terminal; and
at least one driving circuit, each one of the at least one driving circuit having an input terminal connected with the output terminal of the controller and an output terminal connected with the at least one photoelectric element and driving one of the at least one photoelectric element to generate light.

7. The device as claimed in claim 6, wherein
the light source module has six photoelectric elements longitudinally arranged as three rows in a triangular form,
the length of the bottom side of the triangle is 4 cm and the height of the triangle is 2.235 cm, and
the at least one photoelectric element is selected from invisible laser diode, near-infrared diode or light-emitting diode.

8. The device as claimed in claim 7, wherein the controller is externally connected with an oscillator and is embedded with a timer and a task mode switching process to provide at least one task mode for user to select,
wherein each one of the at least one task mode has steps of:
controlling the light generated by the at least one photoelectric element at a frequency and continuously illuminating the light for a period of time; and
controlling the light generated by the at least one photoelectric element at another frequency and further continuously illuminating the light for a period of time.

9. The device as claimed in 8, wherein
the control module further has an input unit connected with the input terminal of the controller, and
the output terminal of the controller is connected with a display driving circuit connected with a display for displaying data and function options and generating background light with different colors.

10. The device as claimed in claim 6, wherein the bearing member takes a form of a wristband mounted around an arm of a user so that the lighting end of each one of the at least one photoelectric element faces an inner side of the arm.

11. The device as claimed in claim 7, wherein the bearing member takes a form of a wristband mounted around an arm of a user so that the lighting end of each one of the at least one photoelectric element faces an inner side of the arm.

12. The device as claimed in claim 8, wherein the bearing member takes a form of a wristband mounted around an arm of a user so that the lighting end of each one of the at least one photoelectric element faces an inner side of the arm.

13. The device as claimed in claim 9, wherein the bearing member takes a form of a wristband mounted around an arm of a user so that the lighting end of each one of the at least one photoelectric element faces an inner side of the arm.
